(19) United States
(12) Patent Application Publication
(10) Pub. No.: US 2011/0195497 A1
(43) Pub. Date: Aug. 11, 2011

(54) METHOD FOR PURIFYING MESENCHYMAL STEM CELLS

(76) Inventors: Claudia Lange, Hamburg (DE); Axel Rolf Zander, Hamburg (DE)

(21) Appl. No.: 12/946,648

(22) Filed: Nov. 15, 2010

Related U.S. Application Data
(62) Division of application No. 10/567,021, filed on Jul. 27, 2006, now abandoned.

(30) Foreign Application Priority Data
Aug. 6, 2003 (DE) 103 36 152.9

(51) Int. Cl. C12N 5/0775

(52) U.S. Cl. 435/366

(57) ABSTRACT

The invention relates to a method for purifying mesenchymal stem cells, producing cells corresponding to prior art, the cells obtained being characterised by an increased proliferation capacity while maintaining the multipotency and typical antigen characteristics. To this end, mesenchymal stem cells that can be multiplied in a significantly improved manner compared to prior art are first provided and can also be differentiated, after a longer cultivation period, in three mesenchymal directions.

The present invention concerns a method for the purification of mesenchymal stem cells (MSC; CD34 negative, plastic adherent, fibroblastoid cells), delivering cell yields comparable with prior art, but where the cells obtained display enhanced proliferation capacity under simultaneous retention of multipotency and typical antigen characteristics. In this way, for the first time, mesenchymal cells become available which—compared to the prior art—are much better expandable, and which in addition can still be differentiated to three mesenchymal lineages after longer cultivation.

### BACKGROUND

Mesenchymal stem cells (MSCs) are extracted from adult bone marrow. There is a multiplicity of protocols throughout the world for generation of MSCs, and these have differing parameters as to, for example, enrichment, the medium used and the choice of foetal calf serum.

An important step in enrichment of the MSCs is depletion of the cells from the bone marrow, which no longer have the potential for proliferation and differentiation (e.g. erythrocytes and granulocytes) as described for MSCs. A widespread method in haematology is density gradient centrifugation with the isotonic solutions Ficoll® or Percoll®. This separation method is based on each defined cell possessing a certain density and moving during centrifugation in the direction of that density of the separation medium where its isopycnic point lies.

At a density of 1.077 g/ml, mononuclear cells (MNCs) are routinely collected at the threshold between sample and density solution, whereas the erythrocytes and granulocytes are concentrated at the bottom of the tube. This density is also used by many authors for MSC enrichment from bone marrow (Azizi et al., Proc Natl Acad Sci USA 95 (1998): 3908-13; Phinney et al., J Cell Biochem 75 (1999): 424-36; DiGirolamo et al., Br J Maematol 107 (1999): 275-81; Muraglia et al., J Cell Science 113 (2000): 1161-66; Colter et al., Proc Natl Acad Sci USA 97 (2000): 3213-18, Proc Natl Acad Sci USA 98 (2001): 7814-45; Quirici et al., Exp Hematol 30 (2002): 783-91). None of the authors has carried out comparative studies with cells that are enriched via other densities, and it remains unclear to what extent there are cells contained in the population of MSC cells with stimulating or suppressing characteristics. Morphologically, two types are shown by Azizi et al., loc.cit.: flattened and elongated cells. Muraglia et al., loc.cit., demonstrated three clone phenotypes: fibroblast-like stretched cells, large flattened cells and narrow starshaped cells. It is known that cultures containing a large number of flattened cells proliferate more slowly or else gradually stop growing. Later sprouting of flat cells can no longer be influenced.

The number of doublings varies for the authors between 4 and almost 50 (Colter et al., Proc Natl Acad Sci USA 97 (2000): 3213-18; the figure of 50 is however questionable, since cumulatively 10¹³ cells were generated from 20 ml of bone marrow, and even with only one cell as an initial figure, 43 doublings would have been achieved) and depends on the quality of the donor bone marrow. In addition, the details of passages available in prior art need to be interpreted cautiously. Generally 5×10³ cells/cm² are plated and the cells harvested at near confluence and counted. Approximately 2 doublings occur per passage.

Cells purified with d=1.077 g/ml showed in vitro differentiation to the osteogenic, chondrogenic and adipogenic lineage at least in early passages. In a few studies (DiGirolamo et al., loc. cit.) capability of differentiation of the MSCs was investigated to culture termination. Muraglia et al., loc. cit., demonstrated using cloned MSCs that osteogenic differentiation did not disappear even in late passages, but adipogenic and chondrogenic differentiation on the other hand did not remain in all clones until culture end. Adipogenic differentiation is first ceased by the cells, but even so the cells cannot be differentiated into chondrocytes up to the final passages. The capability for osteogenic differentiation seems to be a general feature of MSC and generally does not disappear until the end of cell growth.

Other authors use a density of 1.073 g/ml for enrichment (Majumdar et al., J Cell Physiol 176 (1998): 57-66; Mackay et al., Tissue Engineering 4 (1998): 415-28; Pittenger et al., Science 284 (1999): 143-7; Mosca et al., Clin Orthop Rel Res 379S (2000): S71-90; Koc et al., Bone Marrow Transpl 30 (2002): 215-22; Toma et al., Circulation 105 (2002): 93-8) and the cells are designated “low density”. Pittenger et al., loc. cit., describe the isolated cells as morphologically uniform, but in the figures available in the publication flattened cells can also be seen, whose function receives no further comment.

The cells enriched via a density of 1.073 g/ml were positive in three differentiation assays (adipogenic, osteogenic and chondrogenic differentiation) without spontaneous differentiation. Proliferation capability in these cells too is dependent on carefully selected sera. After 2 passages 5-37.5×10⁷ cells are generated, corresponding to data with conventionally separated cells, therefore implementing no advantage for this method.

A third group of authors uses the very complex method of a preformed continuous gradient of 70% Percoll® for bone marrow separation (Lennon et al., In Vitro Cell Dev Biol 32 (1996): 602-11; Bruder et al., J Cell Biochem 64 (1997):278-94; Jaiswal et al., J Cell Biochem 64 (1997):295-312; Fleming et al., Developm Dynamics 212 (1998):119-32; Liechty et al., Nat Med 6 (2000):1282-6). After centrifugation, the first 25% are used as “low density” cells for generation of MSC and pooled density is given as 1.030 g/ml, but for this value, reworking with the available data cannot be reproduced.

The disadvantage of the last mentioned method consists also in the fact that preparation of continuous gradients is costly in terms of time and materials, and there are many laboratories that cannot perform it (a centrifuge with 20,000 g is required). The cells purified with this method also retain, like those described above, their osteogenic differentiation potential during all subcultivations and are positive for MSC specific surface antigens (Bruder et al., loc. cit.). With increasing length of cultivation an increase in the flat, spread-out phenotype is also reported, the cells accumulate debris and stress fibres (Actin), until the culture finally degenerates completely (Bruder et al., loc. cit.).

From the results known to prior art, it is clear that sprouting of less mitotic cells cannot be prevented with any separation method. Rather the cultivation conditions play a large role here. What is needed here is careful selection of the population with most evident proliferation features.

The task of the present invention is therefore to provide a new method for purification of mesenchymal stem cells that does not display the disadvantages known from prior art. In particular the aim of the purification is to minimise the number of flattened cells at the start of culture, since what is known of these cells is that they proliferate more slowly or else gradually stop growing.

As the solution a method is suggested, where mesenchymal stem cells are isolated from bone marrow by means of density gradient centrifugation, but where the cells are isolated from a fraction having a lower density as compared to prior art, that is of <1.073 g/ml, and preferably of ≦1.070 g/ml.

1.050 g/ml to 1.070 g/ml are preferred, with a density of 1.068 g/ml being particularly preferred.

Surprisingly, it was discovered that when using the density of the invention of <1.073 g/ml, and particularly with 1.068 g/ml, cells can be enriched, which—in comparison to cells that were isolated at higher densities—possess enhanced capacity for proliferation but retain the multipotency and typical antigen characteristics. Morphologically, the mesenchymal stem cells of the invention display a fibroblastoid shape for an extended period of time before culture stops.

According to a particularly preferred embodiment, the invention concerns the use of a solution of Ficoll® or Percoll® of 1.068 g/ml density for performing a density gradient centrifugation for isolation of mesenchymal stem cells from bone marrow.

The subject of the invention is also the mesenchymal stem cells (or a preparation that contains exclusively or predominantly—i.e. at least 70%, 80% or preferably at least 90%—these cells) obtained according to the method described, as well as pharmaceutical preparations containing these cells.

The cells according to the invention express the typical surface markers of mesenchymal stem cells (CD90, CD105, CD59). On investigation of expression over many passages to culture end and with increasing length of the period of cultivation, a reduction (from >90% to approximately 60% on average for higher passages) in the expression of positive markers such as CD90 and CD105 is found and no increase in the expression of haematopoietic markers such as CD45 and CD34. The reduction in the expression of mesenchymal markers correlates with the ageing of the cells described (cf. FIG. 6a as against 6b).

As part of the invention, the cells obtained according to the method described were thinly sown (approximately 500 cells/cm²) and by the next passage doubled in number by approximately 3.3 times more than cells that had been isolated according to conventional methods and correspondingly thickly sown (approximately 5,000 cells/cm²) (for these a value of 2 is given). In all, according to the invention, up to 45 doublings were achieved.

All fractions investigated were able to be differentiated by the end of culture into the three mesenchymal lineages investigated:
Differentiation into osteoblasts (osteogenic differentiation; induction according to

Jaiswal et al., loc.cit.) remained uninfluenced by the increasing number of passages. Towards the end of culture, the cells differentiated at times spontaneously into calcium-secreting cells, a fact that underlines this insight.
Adipogenic differentiation (induction according to Pittenger et al., loc.cit.), on the other hand, decreased with increase in number of passages and could ultimately only be detected in individual cells (from approx. 50% after two weeks of differentiation induction to around 1-2% at higher passages).
Differentiation into chondrogenic lineage (induction modified according to Shakibaei et al., Cell Biol Internat 21 (1997): 75-86) also decreased with the increase in number of passages, but not to the same extent as adipogenic differentiation (from approximately 90% after one week of differentiation induction to approximately 15% at higher passages), and one part (approximately 10-20%) of the cells of all fractions displayed typical proteoglycan staining. In cells of higher densities (i.e. from ≧1.077), this chondrogenic differentiation capability was less pronounced than in those of lower densities (i.e. <1.077 g/ml).

With the method of the invention for isolation of mesenchymal stem cells, any (isotonic) gradients may be used, such as, for example, Ficoll® gradients, which involve sucrose cross-linked with epichlorhydrine with a high degree of branching.

As a separation medium, the use of Percoll®, which consists of silica gel particles coated with polyvinylpyrrolidone, and which is not toxic to cells, is especially preferred. It can easily be diluted with buffered salt solutions to the required density without the pH value and osmolality being changed. Ficoll® (a hydrophilic polymer) is indeed also suitable, but repeated dilution involves adjustment of pH value and osmolality.

For separation of bone marrow therefore, by way of example, a discontinuous Percoll® gradient with densities of 1.050 to 1.100 g/ml is prepared. After centrifugation each fraction with a characteristic isopycnic point can be individually removed and investigated. On continuous gradients, on the other hand, exact isopycnic characterisation is not possible and a mix of cells of differing densities is obtained.

The gradient consists, for instance, of 6 defined densities. Each fraction (F1=lowest density, F6=highest density) is investigated for morphology and proliferation potential, expression of MSC-typical markers and multipotency in 3 differentiation assays.

In early passages the “low-density” fractions F1 to F3 (density 1.050 to 1.068 g/ml) consist predominantly of elongated spindle-shaped cells (cf. FIG. 1, F3 in passage 2), whereas in F5 and F6, already at the start of culture, increasingly flat, spread-out cells occurred. F4 with density of 1.077 g/ml (this density is used for separation of MNC) contains with increasing passages more of these large, flat cells, whereas F5 and F6 with densities of 1.088 and 1.100 g/ml display an increased number of flat cells already during primary culture (═PO).

The invention concerns in particular a method for isolating mesenchymal stem cells from bone marrow using density gradient centrifugation where an isotonic solution of Percoll® is used for performing density gradient centrifugation, and where the cells are isolated from a fraction having a density of around 1.068 g/ml.

The method of the invention for the isolation of mesenchymal stem cells can either be performed as part of individual single therapy at the place or clinic where the patient is being treated, but it is practicable to perform the method and subsequent stem cell therapy at larger centres (Good Manufacturing Practice centres; GMP centres), since by doing this a consistent quality standard can be guaranteed. It is also conceivable that there could be enrichment not only of the patient's mesenchymal stem cells that are isolated from his own bone marrow donation (autologous MSCs), but that also allogenic cells, i.e. from other bone marrow donors and other voluntary donors, may be considered, but where these should be understood both as typified and as non-typified allogenic bone marrow donations.

The subject of the invention is, moreover, a method for the manufacture of a pharmaceutical preparation containing mesenchymal stem cells, for which a previously mentioned method for the isolation of mesenchymal stem cells from bone marrow using density gradient centrifugation is performed, and the isolated stem cells are formulated if necessary with pharmaceutically acceptable excipients and carriers.

Moreover, it is also conceivable that as part of the commercial use of the invention for performing the method of isolation of MSCs the required reagents and aids be made available, by way of example in the form of kits containing an isotonic solution of e.g. Ficoll® or Percoll® of density 1.068 g/ml. Alternatively the kits may also contain several isotonic solutions of e.g. Ficoll® or Percoll® of differing density. The solutions of differing density are, by way of example, in the region of 1.050 g/ml to 1.100 g/ml. In accordance with a particular embodiment, the solutions are Percoll® solutions of density 1.050 g/ml, 1.063 g/ml, 1.068 g/ml and 1.070 g/ml. In accordance with one embodiment of the invention, the kits may, if necessary, contain other aids and/or reagents required for the implementation of the method, such as, for example, containers, centrifuge tubes, culture dishes and the like.

The invention is illustrated below by means of examples:

### EXAMPLES

### Materials

As starting solution for manufacture of the discontinuous density gradient Percoll® (Biochrom, Berlin) of density 1.124 g/ml is used. Dilutions of the starting solution using PBS (phosphate-buffered saline without calcium and magnesium ions, Gibco) for the desired densities are calculated using the following formula:

$$V [ \% ] = \frac{( {D}^{′} - D \% ) \times {10}^{2}}{{D}^{′′} - D \%}$$

### Where:

D′ Desired final density (g/ml)
D″ High initial density (g/ml)
D % Density of the iso-osmolar diluent solution (g/ml)
V % Percentage volume for starting solution with high density.

In this way, separation solutions of the following densities were prepared: 1.050, 1.063, 1.068, 1.077, 1.088 and 1.100 g/ml.

### Example 1

### Performing MSC Isolation

5 ml of each of the individual densities were carefully layered into a 50 ml Falcon tube. 10 ml of bone marrow were diluted with 10 ml PBS and carefully layered onto the gradient.

In parallel
a) 1 ml of bone marrow, diluted with 1 ml PBS, was layered onto 3 ml of Ficoll® of density 1.077 g/ml (designated SC stem cells) in a 15 ml Falcon tube as control, and
b) Every 1 ml of bone marrow, diluted with 1 ml of PBS, was layered onto 3 ml Percoll of density 1.068 g/ml (designated LD=low density) or 1.077 g/ml (designated MNC=mononuclear cells) each in a separate 15 ml tube as control.

All tubes were centrifuged at room temperature for 20 min at 800 g without brake. The plasma mixed with PBS was removed from the tube and each fraction transferred into a separate tube. After being washed twice with PBS for 10 minutes at 400 g, the erythrocytes contained in F4 to F6 were removed using haemolysis buffer, the cells were washed again and taken up in DMEM/LG cultivation medium (Dulbecco's Modified Eagle Medium/low glucose, Gibco)+1% penicillin/streptomycin+10% selected foetal calf serum, and then counted in a Neugebauer chamber using Trypan Blue. 1×10⁷ cells are sown on a culture surface of 25 cm² (T25, Greiner). If there are less than 10⁷ cells in a fraction, small culture containers, such as 6-well plates, for instance, are used, corresponding to the number of cells. To detect CFU-Fs (colony-forming unit fibroblasts; described in terms of identifier of proliferation capability of individual fractions in: DiGirolamo C et al., British J. Haematology (1999), 107:275-281) 10⁶ cells of each fraction, as well as of the LD and MNC control cells, are each sown in a separate well of a 6-well plate in 3 ml of medium. The cells are incubated in an incubator at 37° C. and 5% CO₂. After 3 days the non-adherent cells are removed, the culture containers washed with PBS and filled with new medium.

The cells are fed twice weekly by changing the medium and incubated to an 80-90% confluence (visual assessment by microscope). At this point the culture is designated PO as the primary culture. For passaging, all of the medium is removed, the culture area washed with PBS, incubated for 5 minutes with 0.25% Trypsin/EDTA and then resuspended with the addition of medium and counted. 500 cells/cm² are sown in new T25 or T75 to continue culture and now designated P1. The CFU-Fs are washed with PBS after incubating for 14 days and stained with 1% crystal violet.

### Example 2

### Differentiation Experiments

For differentiation experiments 6×10³ cells per well are sown across a 24-well plate, 4 wells each being for induction of osteogenic and adipogenic differentiation.

After reaching confluence, adipogenic differentiation is induced, as described in Pittenger et al. 1999 (loc. cit.). For this, the medium 1 μM of dexamethasone+0.5 mM isobutylmethylxanthine+100 μM indomethacin+10 μM of insulin are added to the medium, and the cells are incubated for 3-4 days. For one day the cells with medium are incubated only with insulin for purposes of conservation. Control wells are each cultivated without these additions, for identification of any spontaneous differentiations that might arise. This cycle of induction and conservation is repeated six times. Subsequently the cells are washed with PBS, fixed for 10 minutes with 4% formalin, washed briefly with 50% ethanol and stained for 15-30 minutes with Sudan Red B. After being briefly washed with 50% ethanol they are counter-stained with haemalaun for 5 minutes, irrigated for 1 minute with tap water and then preserved using liquid paraffin.

For osteogenic differentiation, confluent cultures are induced, as described in Jaiswal et al. 1997 (loc. cit.). For this, the cultures are incubated with a medium where 10⁻⁷ M dexamethasone+50 μM ascorbic acid+10 mM β-glycerol phosphate have been added, and this medium is replaced after 3-4 days. The control cultures receive medium without induction components. After 2-3 weeks the mineral deposits of calcium are stained using the von Kossa method (von Kossa, J. et al. (1901) Beit. Path. Anat. 29: 163). For this, the cells are washed with PBS, fixed for 10 minutes with 4% formaldehyde, washed once with PBS and twice with distilled water and air dried. Then they are stained for 10 minutes with silver nitrate under UV light, washed two to three times with distilled water, counter-stained for 1 minute with haemalaun and, after irrigation with tap water, covered in liquid paraffin.

Chondrogenic differentiation takes place with modification according to the method of Shakibaei et al. 1997 (loc. cit.). For this, 3×10⁴ cells are taken up in an Eppendorf tube in 20 μl of 2% alginate. The alginate cell suspension is dropped in 0.1M CaCl₂ into 6-well plates and gels there for 10 minutes at room temperature. After being washed three times with 0.15 M NaCl, it is washed twice with the medium, and the alginate balls are incubated in the medium for 7 days in the incubator at 37° C. and 5% CO₂ changing the medium once or twice. The alginate balls are fixed in toto for 1 hour in 10% formalin at room temperature, washed for 5 minutes in 2% acetic acid and stained for 24 hours at room temperature in Alcian Blue solution. Here, specifically the proteoglycans that are formed as a matrix by the cells are stained. After being washed 3 times in distilled water, the alginate balls are each dehydrated for 10 minutes through an alcohol series increasing to 90% ethanol, dehydrated for 5-10 minutes in xylol, and embedded in Entellan under light pressure.

### Example 3

### Phenotypical Analysis for Surface Markers

The cells from each passage are again subjected to phenotypical analysis for surface markers. For this the following antibodies were used: CD34-PE (phycoerythrin), CD45-PE, CD90-FITC (fluoroisothiocyanate), CD105-FITC, CD59-FITC and the corresponding isotype controls: mouse IgG1-PE, mouse IgG1-FITC, and mouse IgG2a-FITC.

At least 5-10×10⁴ cells were incubated with the number of antibodies specified by the manufacturer in 50 μl FACS buffer (PBS+2% FCS+0.1% sodium azide; FCS=foetal calf serum) for 20 minutes at room temperature, and then washed with FACS buffer. The stained cells are resuspended in 3-400 μl FACS buffer and subjected to analysis on a FACScan/Becton Dickinson. There, the settings for forward and side scatter characteristics, as well as fluorescence, are performed with the isotype control. Evaluation is carried out using the CellQuest software from Becton Dickinson.

Even in poorly growing cultures or poorly growing fractions the cells of the invention display at least 20 doublings and must be ascribed to the very carefully selected FCS. Initially 500 cells/cm² were plated out. Up to the 80-90% confluence the cells double, depending on the passage, around 2-6 times. On FCS selection, growth, phenotype and differentiation into three lineages were analysed. Growth curves were generated up until passage 4. In passage 4 the phenotype and differentiation into three lineages (osteogenic, chondrogenic and adipogenic lineage) were analysed. If the phenotype and differentiation potential were the same for different sera, priority was accorded to more rapid growth.

### Results

Comparison of the proliferation rates of the individual fractions showed that cells that were separated with densities of 1.05 to 1.068 g/ml (corresponds to F1 to F3) achieve more cell divisions (FIG. 2) and therefore more doublings (FIG. 3) up to the termination of culture than cells of higher densities. SC designates the accompanying control of cells purified using Ficoll®. The doublings shown in FIG. 3 are calculated from the cell number of the sample from FIG. 2. Depending on the quality of the donor bone marrow, the proliferation characteristics of the defined fractions are not always identical, but always display prominence of the “low density” fractions. The difference in doubling rates between F3 as “low density” cells and F4 as MNCs can amount to up to 5 doublings, i.e. for instance 10¹³ cells would become 3.2×10¹⁴ cells.

These results are confirmed by analysis of CFU-Fs of the individual fractions and comparison of LD and MNC cells (FIG. 4). A large number of CFU-Fs can be clearly seen in the fractions F1 to F3 and heavily reduced numbers in F4. In F5 and F6 there are hardly any CFU-Fs any more. In the LD cells, on the other hand, the highest number of these colony forming cells is to be found, and clearly more than in the individual fractions F1, F2 and F3, as also than in the MNC cells.

The analyses relating to phenotype of the MSCs separated into fractions showed populations that are negative for the haematopoietic markers CD45 and CD34 (not illustrated), but positive for CD90 (Thy-1, marker for early progenitor cells), CD105 (endoglin, specific marker for MSC) and CD59 (Sca-1=Stem cell antigen homologue, marker for earlier stem cells, not illustrated). In the FACS analysis the MSCs can be subdivided into two populations: a small population R1, with approximately 2-5%, consists of small, barely granulated cells; and a prominent population R2 that consists of highly granulated cells (FIGS. 5a and 6a, the left histogram in each case). The R1 cells, both from the fractions of low density (FIG. 5a, b) and from those of higher density (FIG. 6a, b), are negative for CD90 and CD105 (small peak in the black curve; grey: isotype control), whereas the main population R2 is positive for both markers.

With this antigen profile the R1 cells appear rather to represent a highly immature population. The number of cells in the R2 population decreases as time of cultivation increases. In the histograms, more cells appear in R1, which, however, is due to an increase in apoptotic (dying) cells and debris. As cultivation progresses, there is an evident decrease in the number of positive cells for both markers shown. Reduction in CD90 and CD105 positive cells is more prominent in fractions of higher density (FIG. 6b as against FIG. 6a) than in fractions of lower densities (FIG. 5b as against FIG. 5a). If we correlate the reduction in expression of MSC-typical markers with cell capability for osteogenic lineage differentiation, then this differentiation does not seem to depend on expression of the markers on all cells.

### DESCRIPTION OF THE FIGURES

FIG. 1: Spindle shaped MSCs in fraction 3 in the 2^{nd} passage.

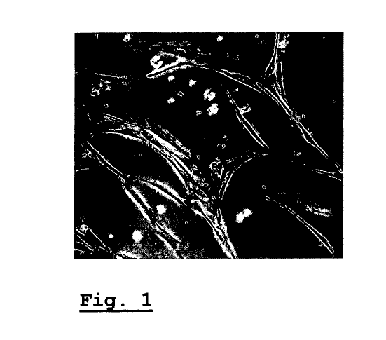

FIG. 2: Relative cell numbers based on an example from 3 experiments.

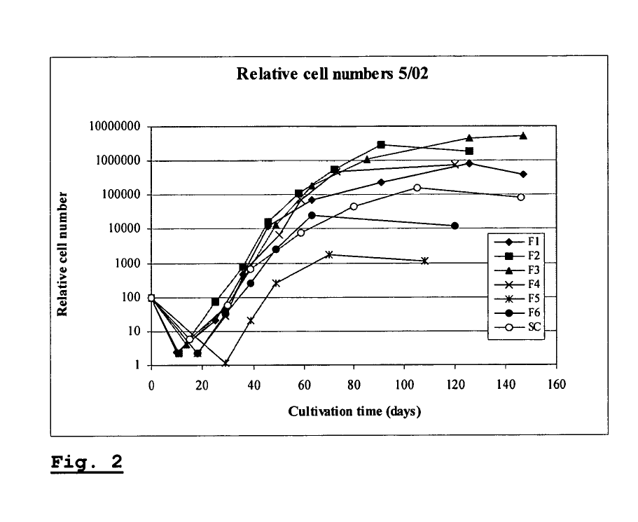

FIG. 3: Doublings of individual fractions of an example from 3 experiments.

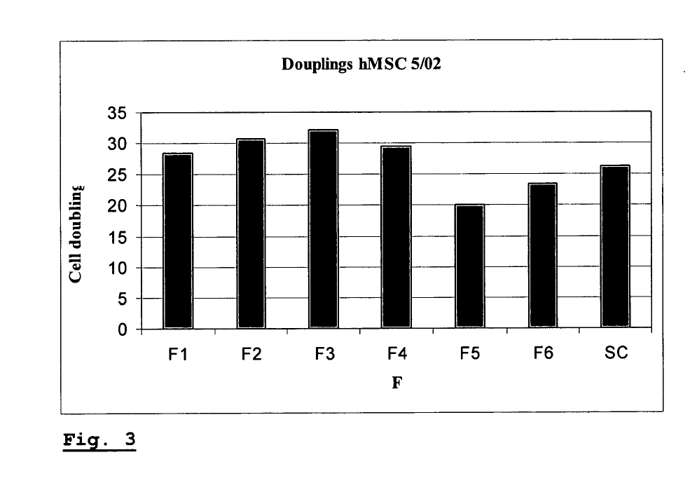

FIG. 4: CFU-Fs of the individual fractions F1 to F6 in comparison with LD and MNC cells of an example from 5 experiments.

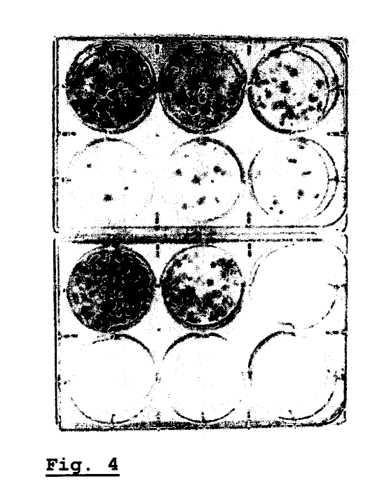

FIG. 5a: Scatter characteristics and expression of surface markers on an example of MSCs from fraction F3 in the 2^{nd} passage.

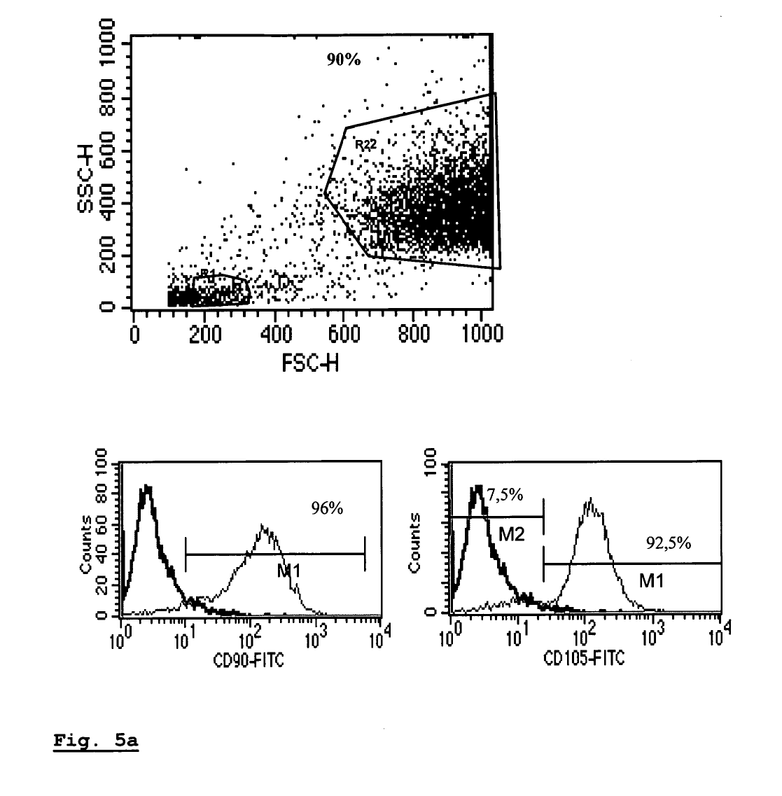

FIG. 5b: Scatter characteristics and expression of surface markers on an example of MSCs from fraction F3 in the 7^{th} passage.

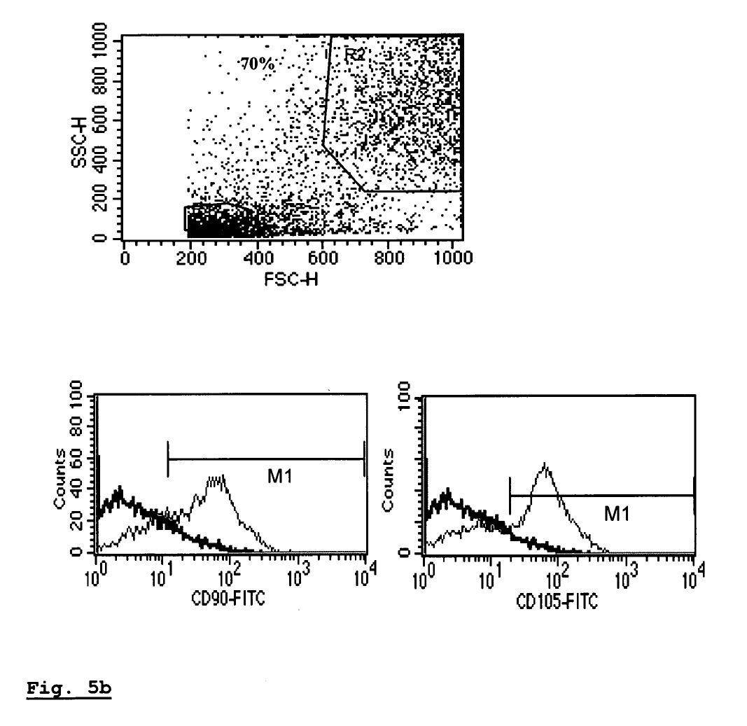

FIG. 6a: Scatter characteristics and expression of surface markers on an example of MSCs from fraction F6 in the 2^{nd} passage.

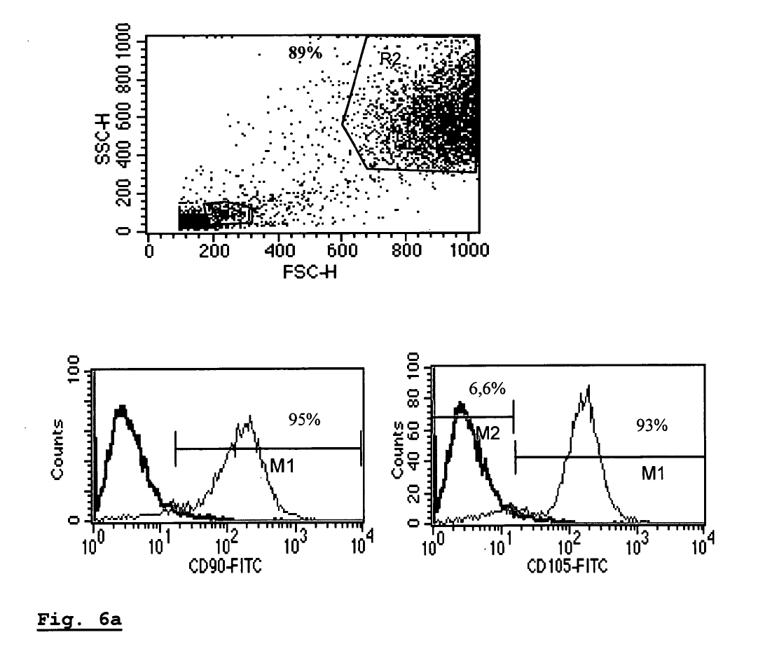

FIG. 6b: Scatter characteristics and expression of surface markers on an example of MSCs from fraction F6 in the 7^{th} passage.

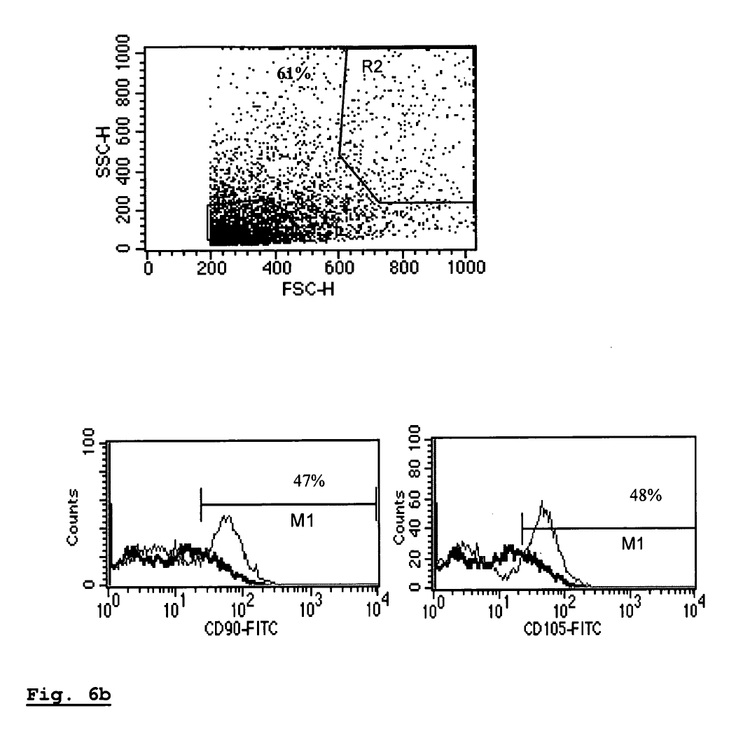

What is claimed is:

1. A method of isolating a population of human mesenchymal stem cells comprising performing density gradient centrifugation on human bone marrow; and
isolating human mesenchymal stem cells from a fraction having a density of 1.050-1.070 g/ml, wherein the population of mesenchymal stem cells does not express CD34 on their cell surfaces.

2. The method according to claim 1, wherein said density is 1.068 g/ml.

3. The method of claim 1, wherein the population of mesenchymal stem cells make up at least 70% of a preparation in which the cells are isolated.

4. The method of claim 1, wherein the population of mesenchymal stem cells make up at least 80% of a preparation in which the cells are isolated.

5. The method of claim 1, wherein the population of mesenchymal stem cells make up at least 90% of a preparation in which the cells are isolated.
